(19) United States
(12) Patent Application Publication
(10) Pub. No.: US 2021/0324009 A1
(43) Pub. Date: Oct. 21, 2021

(54) ANTI-BACTERIAL CALCIUM-DEPENDENT ANTIBIOTIC (CDA) ANALOGS AND METHODS OF TREATING BACTERIAL INFECTIONS

(71) Applicant: THE UNIVERSITY OF HONG KONG, Hong Kong (CN)

(72) Inventors: Xuechen LI, Hong Kong (CN); Delin CHEN, Hong Kong (CN)

(21) Appl. No.: 17/220,177

(22) Filed: Apr. 1, 2021

Related U.S. Application Data
(60) Provisional application No. 63/011,510, filed on Apr. 17, 2020.

(51) Int. Cl. C07K 11/02; A61P 31/04

(52) U.S. Cl. CPC C07K 11/02; A61P 31/04

(57) ABSTRACT

Provided herein are calcium-dependent antibiotics (CDAs), as a novel therapeutic target for treating bacterial infections. The present invention also relates to pharmaceutical compositions comprising such compounds, and to methods of use thereof for combating bacteria and treating bacterial infections.

The present application claims priority to U.S. provisional application Ser. No. 63/011,510, filed Apr. 17, 2020 which is incorporated by reference in its entirety.

### 1. FIELD

Described herein are calcium-dependent antibiotic (CDA) analogues, methods of preparation thereof, and use of such compounds for treating bacterial infections, in particular for infections with resistance to conventional antibiotic treatment.

### 2. BACKGROUND

The rapid emergence and wide spread of bacterial resistance to existing antibacterial drugs becomes an urgently global threat to human health. Cyclic peptides have been an important class of modalities as antibacterial agents, such as vancomycin, bacitracin, colistin/polymyxin, gramicidin and daptomycin all being clinically used. Some new cyclic peptides with promising antibacterial profiles have been discovered in recent years, including teixobactin, malacidins, cadaside, darobactin, which hold promise for future clinical applications. Among all these antibacterial cyclic peptides, daptomycin, A54145, malacidins and cadaside are grouped into calcium dependent antibiotics, which require the Ca²⁺ in serum to exert their antibacterial acts.

The calcium-dependent antibiotics (CDAs) were discovered from Streptomyces coelicolor A3(2) in 1983 (FIG. 1). It comprises an exocyclic tail containing a serine and N-terminal fatty acid side chain (a trans-2,3-epoxyhexanoyl moiety), along with ten-amino acid residue decapeptide lactone core. However, the antibacterial activities of these CDAs are not very potent. Based on complete genome sequence of CDAs' producer Streptomyces coelicolor A3, the biosynthesis of CDAs has been established and later engineered to produce CDAs' analogues. Due to bioengineering restriction, the modifications were limited to the specific amino acid residues, and none of the generated analogues showed comparable or improved antibacterial activity compared to the parent compound.

CDA3a features an unusual trans-2,3-epoxyhexanoyl exocyclic tail, which together with three non-proteinogenic amino acid residues including Z-dehydrotryptophan at position 11, D-4-hydroxyphenylglycine (D-Hpg) at position 6 and (2R,3S)-3-hydroxyasparagine (3-OHAsn) at position 8, influences the topology of the molecule, potentially contributing to its bioactivity.

There is an ongoing and unmet need in the art to identify new compounds acting as anti-bacterial agents. In addition, there is a need to combat the growing problem of bacterial resistance to anti-bacterial agents.

### 3. SUMMARY OF THE INVENTION

Certain embodiments of the invention relate to a new class of cyclic peptides as antibiotics. The antibiotics of the invention are derivatives of CDA3a. CDA3a has a unique structural motif, as shown in FIG. 1. The structural motif of CDA3a is modified to develop CDA3a analogues, which represent the antibiotics of the invention. The antibiotics of the invention exhibit antibacterial and therapeutic properties superior to those of CDA3a. The novel antibiotics described herein offer substantial promise to combat the serious problem of antibiotic-resistant bacteria.

In specific embodiments, the antibiotics of the invention have a structure of Formula I, II, provided below:

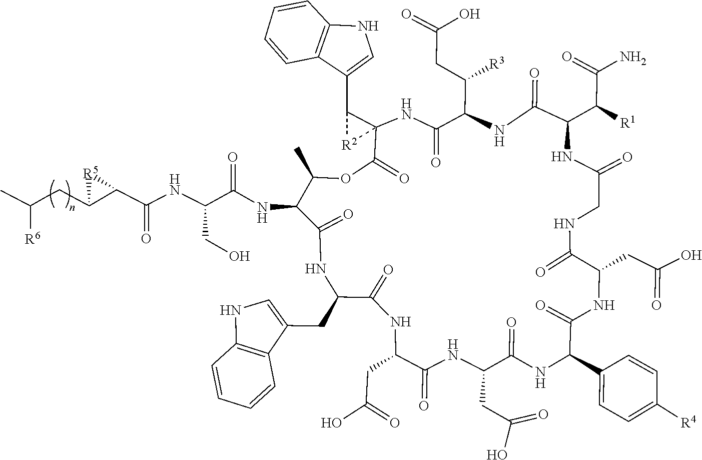

In preferred embodiments, each of R₁ to R₄ are the chemical substituents of the side chain of the amino acid in the cyclic core, for example, R¹ could be H, OH or OPO₃H₂, R² could be H,H or a π bond, R³ could be H or CH₃, R₄ could be H, OH or halogen. R⁵ could be O or a π bond, R⁶ could be H or methyl, and n is 3-12.

Pharmaceutical compositions comprising the antibiotics of the invention and pharmaceutically acceptable carriers or excipients are also provided. Further provided are methods of treating an infection by an agent in a subject by administering to the subject the antibiotics described herein.

Provided herein is an antibacterial compound represented by the structure of formula

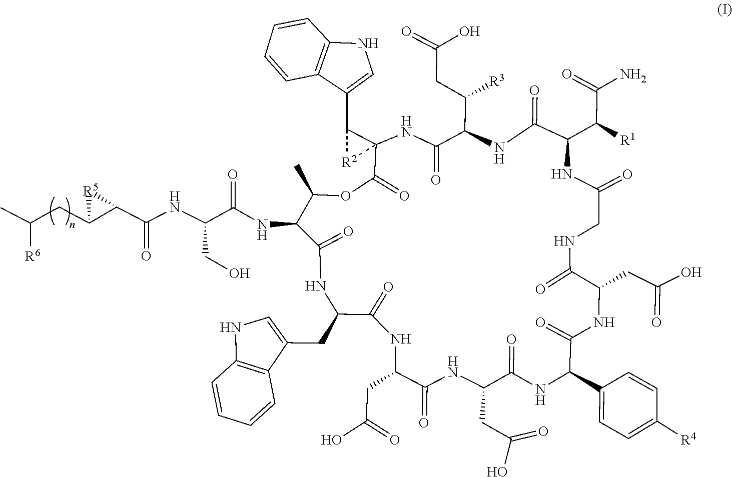

wherein:
R¹ is selected from the group consisting of H, OH and OPO₃H₂;
R² is selected from the group consisting of H,H and a π bond;

R³ is selected from the group consisting of H and CH₃;

R⁴ is selected from the group consisting of H, OH and halogen;

R⁵ is selected from the group consisting of 0 and a π bond

R⁶ is selected from the group consisting of H and CH₃.

n is an integer from 3-12;

and pharmaceutically acceptable salts thereof.
In certain embodiments, the R¹ is H.
In certain embodiments, the R¹ is OH.
In certain embodiments, the R² is H,H.
In certain embodiments, the R² is a π bond.
In certain embodiments, the R³ is H.
In certain embodiments, the R⁴ is OH.
In certain embodiments, the R⁵ is O.
In certain embodiments, the R⁵ is a π bond.
In certain embodiments, the n is an integer from 3 to 12.
In certain embodiments, the R⁶ is H.
In certain embodiments, the R⁶ is CH₃.
In certain embodiments,
(a) R¹ is 3(S)—OH, R² is a π bond; R³ is H, R⁴ is OH, and R⁵ is a π bond, R⁶ is H, wherein n is 5; [Δ^{2,3}-C10-CDA3a]
(b) R¹ is 3(S)—OH, R² is a π bond; R³ is H, R⁴ is OH, and R⁵ is a π bond, R⁶ is CH₃, wherein n is 6; [Δ^{2,3}-C12-CDA3a]
(c) R¹ is 3(S)—OH, R² is a π bond; R³ is H, R⁴ is OH, and R⁵ is a π bond, R⁶ is CH₃, wherein m is 7; [Δ^{2,3}-C13-CDA3a]
(d) R¹ is 3(S)—OH, R² is a π bond; R³ is H, R⁴ is OH, and R⁵ is a π bond, R⁶ is CH₃, wherein m is 8; [Δ^{2,3}-C14-CDA3a]
(e) R¹ is 3(S)—OH, R² is a π bond; R³ is H, R⁴ is OH, and R⁵ is a π bond, R⁶ is CH₃, wherein m is 9; [Δ^{2,3}-C15-CDA3a]
(f) R¹ is 3(S)—OH, R² is a π bond; R³ is H, R⁴ is OH, and R⁵ is a π bond, R⁶ is CH₃, wherein m is 10; [Δ^{2,3}-C16-CDA3a]
(g) R¹ is 3(S)—OH, R² is a π bond; R³ is H, R⁴ is OH, and R⁵ is O, R⁶ is H, wherein n is 5; [epoxyC10-CDA3a]
(h) IV is 3(S)—OH, R² is a π bond; R³ is H, R⁴ is OH, and R⁵ is O, R⁶ is CH₃, wherein m m is 8; [epoxyC14-CDA3a]
(i) IV is 3(R)—OH, R² is a π bond; R³ is H, R⁴ is OH, and R⁵ is O, R⁶ is CH₃, wherein m is 8; [epoxy-C14-epi-D-HOAsn-CDA3a]
(j) R¹ is H, R² is a π bond; R³ is H, R⁴ is OH, and R⁵ is O, R⁶ is CH₃, wherein m is 8; [epoxy-C14-CDA4a]; or
(k) IV is 3(S)—OH, R² H, H; R³ is H, R⁴ is OH, and R⁵ is O, R⁶ is CH₃, wherein m is 8; [epoxy-C14-CDA3b]
In certain embodiments, the compound is a CDA-3a antibiotic analogue.
Provided herein is an anti-bacterial pharmaceutical composition comprising a therapeutically effective amount of the compound disclosed herein, and a pharmaceutically acceptable carrier or excipient.
Provided herein is a method of combating bacteria, or treating bacterial infections, comprising the step of contacting the bacteria with a compound described herein or a pharmaceutical composition described herein.
Provided herein is a method of treating a bacterial infection, comprising the step of administering a compound described herein, or a pharmaceutical composition described herein, to a subject in need thereof.
In certain embodiments, the bacteria is a gram-positive bacteria.
In certain embodiments, the bacteria is a gram-negative bacteria.
In certain embodiments, the bacteria is a methicillin-resistant bacteria.
In certain embodiments, the bacteria is a vancomycin-resistant bacteria.
Provided herein is a method of using the compound disclosed herein or a pharmaceutical composition disclosed herein, for use in the treatment of bacterial infections.

### 4. BRIEF DESCRIPTION OF THE DRAWINGS

The disclosure is illustrated in the figures of the accompanying drawings which are meant to be exemplary and not limiting, in which like references are intended to refer to like or corresponding parts, and in which:

FIG. 1A-B. (A) Structures of CDA3a and its congeners. (B) Table for substituents.

FIG. 2A-C. The MIC of epoxyC14-CDA3a and Δ^{2,3}C14-CDA3a against SA ATCC29213 (A) and EF ATCC29212 (B) was assessed at various concentrations of calcium. The highest concentration tested was 32 μg/mL. The cell permeability of CDA3a, epoxyC14-CDA3a and epoxyC10-CDA3a, Δ^{2,3}C12-CDA3a, Δ^{2,3}C13-CDA3a, Δ^{2,3}C14-CDA3a (C), SA ATCC29213 was incubated with the 0.5 μg/mL CDA3a analogues for 0.5 h. Daptomycin act as positive control. Untreated negative also included.

### 4.1 DEFINITIONS

In the present disclosure the following terms are used.

### Chemical Definitions

An “alkyl” group refers to any saturated aliphatic hydrocarbon, including straight-chain and branched-chain alkyl groups. In one embodiment, the alkyl group is a Cl alkyl, namely methyl (CH₃). In another embodiment, the alkyl group has 6-14 carbons designated here as C₆-C₁₄-alkyl. In one embodiment, the C₆-C₁₄-alkyl is a straight chain alkyl. In another embodiment, the C₆-C₁₄-alkyl is a branched chain alkyl. Examples of alkyl groups include, but are not limited to, hexyl, heptyl, octyl, nonyl, decyl and the like.

In some embodiments, the alkyl is part of a fatty acid residue, wherein the fatty acid is represented by the formula RCOOH wherein R may be:

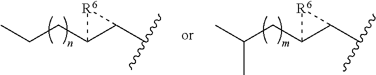

and wherein R⁶ is selected from the group consisting of O and a π bond.

In some embodiments, R is

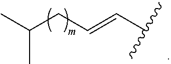

In other embodiments, R is

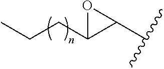

wherein n is an integer from 4 to 12 and m is an integer from 3 to 11. In some embodiments, the fatty acid is a saturated fatty acid. In other embodiments, the fatty acid is an unsaturated fatty acid. In some embodiments, the fatty acid side chain comprises a 2,3-epoxyalkanoyl moiety, comprising a terminal alkyl chain preferably having from 6 to 14 carbon atoms.

The term “halogen” or “halo” as used herein alone or as part of another group refers to chlorine, bromine, fluorine, and iodine. In one embodiment, the halogen is chlorine. In another embodiment, the halogen is bromine. In another embodiment, the halogen is iodine. In another embodiment, the halogen is fluorine.

The term “hydroxy” as used herein alone or as part of another group refers to an OH group.

All stereoisomers of the above compounds are contemplated, either in admixture or in pure or substantially pure form. The compounds can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. Compounds comprising amino acid residues include residues of D-amino acids, L-amino acids, or racemic derivatives of amino acids. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to, carboxylate salts or salts with amine nitrogens, and include salts formed with the organic and inorganic anions and cations. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The term “organic or inorganic cation” refers to counter-ions for the anion of a salt. The counter-ions include, but are not limited to, alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances, the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline or amorphous state of a substance, which can be characterized by particular physical properties such as X-ray diffraction, IR or Raman spectra, melting point, and the like.

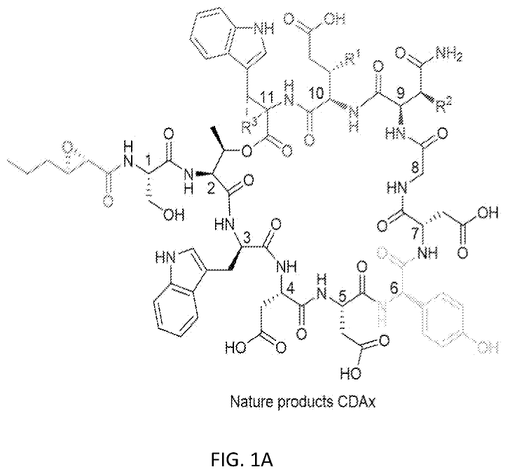

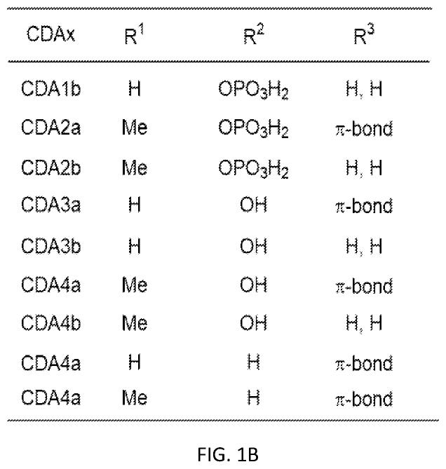

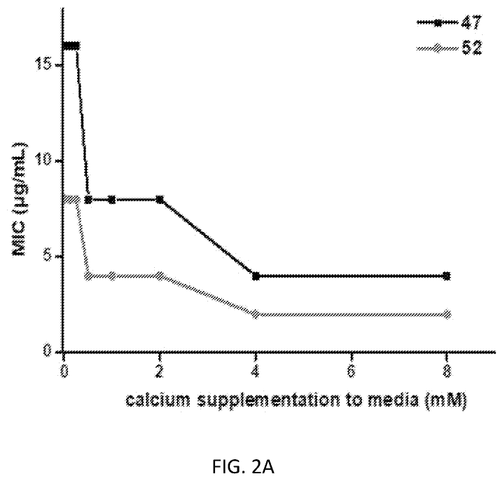

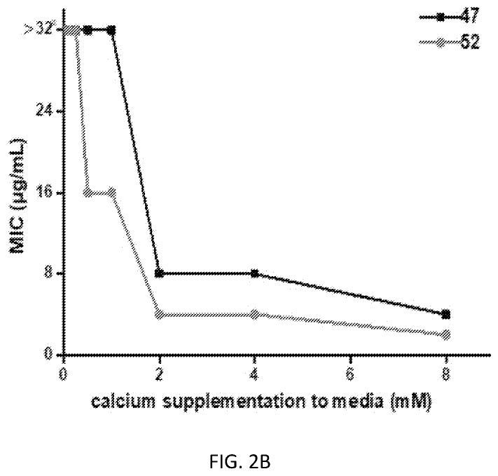

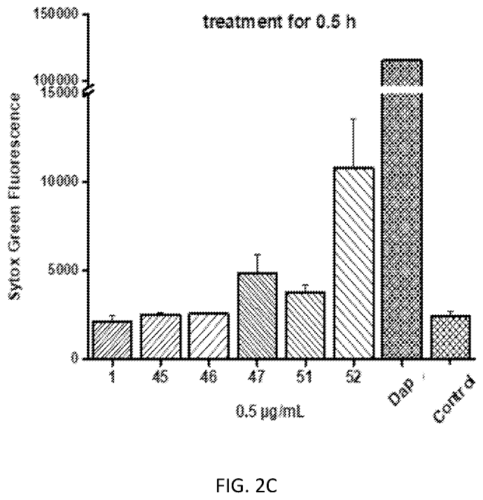

The term “CDA” as used herein refers to a calcium-dependent antibiotic analog. CDAs of formula (I) comprise an exocyclic tail containing a serine and an N-terminal fatty acid side chain comprising a 2,3-epoxyalkanoyl moiety. The family of CDAs include CDA1b, CDA2a, CDA2b, CDA3a, CDA3b, CDA4a, CDA4b, CDA2d, CDA2fa, and CDA2fb. The structures of these compounds are set forth in FIG. 1 and in Hojati et al. Cell Chemical Biology, 2002, Vol 9(11), p. 1175-1187, the contents of which are hereby incorporated by reference.

### 5. DETAILED DESCRIPTION

Embodiments of the subject matter described herein are directed towards a convergent synthesis of naturally occurring calcium-dependent antibiotic CDA3a and its analogues has been developed. The late-stage serine ligation strategy allowed us to readily construct the analogues with the variation on the lipid tail. Among 19 analogues prepared, we identified the analogue 100-500 folds more active than the parent compound against Gram-positive pathogens, which also exhibited antibacterial activity in absence of calcium. The structure-activity relationship and potential mechanisms of action of this type of cyclic peptides were addressed at certain degree by calcium dependent experiment, and cell permeability experiment.

The present disclosure provides a synthetic class of CDA derivatives, in particular CDA3a derivatives having desirable antibacterial activities. The antibiotics of the present invention can be chemically synthesized. The antibiotics of the invention are active against Gram-positive bacteria, including methicillin-resistant Staphylococcus aureus (MRSA), vancomycin-resistant Enterococcus (VRE) and Mycobacterium tuberculosis via synthetic tailoring of CDA3a core structure.

In certain embodiments, the antibiotics of the invention have Formula I:

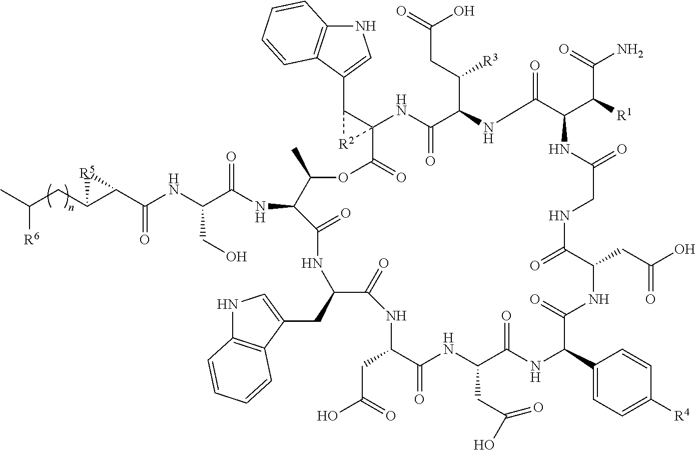

In preferred embodiments, each of R₁ to R₄ are the chemical substituents of the side chain of the amino acid in the cyclic core, for example, R¹ could be H, OH or OPO₃H₂, R² could be H,H or a π bond, R³ could be H or CH₃, R4 could be H, OH or halogen. R⁵ could be O or a π bond, R⁶ could be H or methyl, and n is 3-12.

The antibiotics of the invention can be prepared using solution phase synthesis/solid phase synthesis hybrid method. In such hybrid method, fatty acid side chain in the natural CDA3a (FIG. 1) is replaced by different length carbohydrate side chain. Also, the residues in the cyclic core of CDA3a can be exchanged with other amino acids.

Based on various antibiotics of formulas I to II and different substituents at R₁ to R₅ positions and different fatty acid side chain, the invention provides a range of antibiotics. Exemplary antibiotics based on various substituents were synthesized and characterized.

For example, trans-2,3-epoxyhexanoyl tail of CDA3a of FIG. 1 was replaced with other different length and substituents and the resulting antibiotic exhibited antibacterial activity against methicillin-susceptible S. aureus strain ATCC29213 (MSSA), and a methicillin-resistant S. aureus (MRSA) clinical isolate (MICs: 2-8 μg/mL). The antibacterial activity of this antibiotic against such MSSA and MRSA strains are superior to CDA3a.

Antibacterial activities of additional such examples are provided in Table 1 below:

TABLE 1
Minimal inhibitory concentrations (in μg/ml) of CDA3a and its analogues.

| | MRSA | SA | Streptococcus | Enterococcus |
|---|---|---|---|---|
| Bacterial strain | SA11I4 | ATCC29213 | faecalis SF | ATCCET |
| CDA3a | 512 | 256 | 256 | >1024^{a} |
| Δ^{2,3}C10CDA3a | 8 | 4 | >32 ^{b} | >32 ^{b} |
| Δ^{2,3}C12CDA3a | 8 | 4 | 4 | 4 |
| Δ^{2,3}C13CDA3a | 4 | 2 | >32 | >32 |
| Δ^{2,3}C14CDA3a | 4 | 4 | 8 | 8 |
| Δ^{2,3}C15CDA3a | 4 | 4 | 16 | 16 |
| Δ^{2,3}C16CDA3a | 8 | 8 | 4 | 4 |
| epoxyC10CDA3a | 8 | 2 | >32 | >32 |
| epoxyC14CDA3a | 2 | 2 | 2 | 2 |
| epoxyC14-epi- | 8 | 4 | 4 | 8 |
| HOAsn-CDA3a | | | | |
| epoxyC14-CDA4a | 4 | 4 | 2 | 4 |
| epoxyC14-CDA3b | 4 | 4 | 2 | 8 |

^{a}The highest concentration tested was 1024 μg/mL.
^{b} The highest concentration tested was 32 μg/mL.
MRSA SA11I4, Methicillin-resistant Staphylococcus aureus strain SA11I4; SA ATCC29213, Staphylococcus aureus strain ATCC29213.

As shown in table 1, all these analogues exhibited very potent anti-bacterial activities against MRSA, SA, Streptococcus faecalis and Enterococcus strains. Δ^{2,3}C10CDA3a, Δ^{2,3}C12CDA3a, Δ^{2,3}C13CDA3a and Δ^{2,3}C14CDA3a, Δ^{2,3}C15CDA3a, Δ^{2,3}C16CDA3a (the epoxy was replaced by an double bond in the fatty acid side chain) showed 64-128 times higher activities than CDA3a. expoxyC10CDA3a, epoxyC14CDA3a, epoxyC14-eip-HOAsn-CDA3a, epoxyC14CDA4a and epoxyC14CDA3b also showed 64-256 times higher activities than CDA3a.

Some embodiments of the invention provide methods of treating an infection in a subject by administering the antibiotics of the invention to the subject. In some embodiments, the subject is a plant or an animal, preferably, a mammal, and more preferably, a human. In certain embodiments, the infection is caused by an agent such as, but not limited to, a bacterium, a fungus, a virus, a protozoan, a helminth, a parasite, and combinations thereof.

Accordingly, certain embodiments of the methods of treating an infection comprise administering to a subject a therapeutically effective amount of antibiotics described herein, for example, an antibiotic Formula I, II, thereby treating the infection in the subject.

In particular embodiments, the agent is a bacterium, such as a Gram-positive bacterium or a Gram-negative bacterium. Non-limiting examples of Gram-positive bacteria include Streptococcus, Staphylococcus, Enterococcus, Corynebacteria, Listeria, Bacillus, Erysipelothrix, and Actinomycetes. Non-limiting examples of Gram-negative bacteria include Helicobacter, Neisseria, Campylobacter, Enterobacter, Pseudomonas, Klebsiella, Pasteurella, Bacteroides, Streptobacillus, Leptospira, Salmonella, and Citrobacter.

In some embodiments, the antibiotics of Formula I, II are used to treat an infection by one or more of: Helicobacter pylori, Legionella pneumophilia, Mycobacterium tuberculosis, Mycobacterium avium, Mycobacterium intracellulare, Mycobacterium kansaii, Mycobacterium gordonae, Mycobacteria sporozoites, Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae pyogenes (Group B Streptococcus), Streptococcus dysgalactia, Streptococcus faecalis, Streptococcus bovis, Streptococcus pneumoniae, pathogenic Campylobacter sporozoites, Enterococcus sporozoites, Haemophilus influenzae, Pseudomonas aeruginosa, Bacillus anthracis, Bacillus subtilis, Escherichia coli, Corynebacterium diphtheriae, Corynebacterium jeikeium, Corynebacterium sporozoites, Erysipelothrix rhusiopathiae, Clostridium perfringens, Clostridium tetani, Clostridium difficile, Enterobacter aerogenes, Klebsiella pneumoniae, Pasteurella multocida, Bacteroides thetaiotamicron, Bacteroides uniformis, Bacteroides vulgatus, Fusobacterium nucleatum, Streptobacillus moniliformis, Leptospira, and Actinomyces israelli. In particular, bacterium is methicillin resistant S. aureus or B. anthracis.

In other embodiments, the antibiotics described herein can be used to treat viral infections. Non-limiting examples of infectious viruses that may be treated by the antibiotics of Formula I, include: Retroviridae (e.g., human immunodeficiency viruses, such as HIV-1 (also referred to as HTLV-III, LAV or HTLV-III/LAV), or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses, severe acute respiratory syndrome (SARS) virus); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Birnaviridae; Hepadnaviridae (e.g., Hepatitis B virus); Parvoviridae (parvoviruses); Papovaviridae (papilloma viruses, polyoma viruses); Adenoviridae (most adenoviruses); Herpesviridae (e.g., herpes simplex virus (HSV) 1 and 2, varicella zoster virus, cytomegalovirus (CMV), herpes viruses); Poxviridae (e.g., variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parentally transmitted, i.e., Hepatitis C); Norwalk and related viruses, and astroviruses). In specific embodiments, the antibiotics of Formula I, II are used to treat an influenza virus, human immunodeficiency virus, or herpes simplex virus.

In yet other embodiments, the antibiotics described herein are useful in treating infections caused by protozoans. Non-limiting examples of protozoa that can be inhibited by the antibiotics of Formula I include, but are not limited to, Trichomonas vaginalis, Giardia lamblia, Entamoeba histolytica, Balantidium coli, Cryptosporidium parvum and Isospora belli, Trypansoma cruzi, Trypanosoma gambiense, Leishmania donovani, and Naegleria fowleri.

In certain embodiments, the antibiotics described herein are useful in treating infections caused by helminths. Non-limiting examples of helminths that can be inhibited by the antibiotics of Formula I, II include, but are not limited to: Schistosoma mansoni, Schistosoma cercariae, Schistosoma japonicum, Schistosoma mekongi, Schistosoma hematobium, Ascaris lumbricoides, Strongyloides stercoralis, Echinococcus granulosus, Echinococcus multilocularis, Angiostrongylus cantonensis, Angiostrongylus constaricensis, Fasciolopis buski, Capillaria philippinensis, Paragonimus westermani, Ancylostoma dudodenale, Necator americanus, Trichinella spiralis, Wuchereria bancrofti, Brugia malayi, and Brugia timori, Toxocara canis, Toxocara cati, Toxocara vitulorum, Caenorhabiditis elegans, and Anisakis spp.

In some embodiments, the antibiotics described herein are useful in treating disorders caused by parasites. Non-limiting examples of parasites that can be inhibited by the antibiotics of Formula I, II include, but are not limited to, Plasmodium falciparum, Plasmodium yoelli, Hymenolepis nana, Clonorchis sinensis, Loa boa, Paragonimus westermani, Fasciola hepatica, and Toxoplasma gondii. In specific embodiments, the parasite is a malarial parasite.

In further embodiments, the antibiotics of Formula I, II may be useful to treat disorders caused by fungi. Non-limiting examples of fungi that may be inhibited by the antibiotics of Formula Formula I, II include, but are not limited to, Cryptococcus neoformans, Histoplasma capsulatum, Coccidioides immitis, Blastomyces dermatitidis, Chlamydia trachomatis, Candida albicans, Candida tropicalis, Candida glabrata, Candida krusei, Candida parapsilosis, Candida dubliniensis, Candida lusitaniae, Epidermophyton floccosum, Microsporum audouinii, Microsporum canis, Microsporum canis var. distortum Microsporum cookei, Microsporum equinum, Microsporum ferrugineum, Microsporum flavum, Microsporum gallinae, Microsporum gypseum, Microsporum nanum, Microsporum persicolor, Trichophyton ajelloi, Trichophyton concentricum, Trichophyton equinum, Trichophyton flavescens, Trichophyton gloriae, Trichophyton megnini, Trichophyton mentagrophytes var. erinacei, Trichophyton mentagrophytes var. interdigitale, Trichophyton phaseoliforme, Trichophyton rubrum, Trichophyton rubrum downy strain, Trichophyton rubrum granular strain, Trichophyton schoenleinii, Trichophyton simii, Trichophyton soudanense, Trichophyton terrestre, Trichophyton tonsurans, Trichophyton vanbreuseghemii, Trichophyton verrucosum, Trichophyton violaceum, Trichophyton yaoundei, Aspergillus fumigatus, Aspergillus flavus, and Aspergillus clavatus.

In yet another embodiment, the present invention relates to a method of inhibiting the growth of an infectious agent, the method comprising contacting the agent with a compound described herein, e.g., a compound of Formula I, II, thereby inhibiting the growth of the infectious agent.

### Routes of Administration and Dosage Forms

Certain embodiments of the invention provide pharmaceutical compositions comprising antibiotics of the invention. The pharmaceutical compositions of the invention comprise antibiotics of the invention and pharmaceutically acceptable carriers or excipients.

In certain embodiments, the antibiotics may be administered intramuscularly, subcutaneously, intrathecally, intravenously or intraperitoneally by infusion or injection. Solutions of the antibiotics can be prepared in water, optionally mixed with a nontoxic surfactant. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the antibiotics that are adapted for the extemporaneous preparation of sterile injectable or infusible solutions or dispersions, optionally encapsulated in liposomes. Preferably, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by, for example, the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the antibiotics in the required amount in the appropriate solvent as described herein with various of the other ingredients enumerated herein, as required, preferably followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

The compositions of the subject invention may also be administered orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet.

For oral therapeutic administration, the antibiotics may be combined with one or more excipients and used in the form of ingestible tablets, buccal tablets, troches, capsules, elixirs, suspensions, syrups, wafers, and the like. Such compositions and preparations should contain at least 0.1% of an antibiotic of the present invention. The percentage of the antibiotics of the invention present in such compositions and preparations may, of course, be varied and may conveniently be between about 2% to about 60% of the weight of a given unit dosage form. The amount of the antibiotics in such therapeutically useful compositions is such that an effective dosage level will be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, fructose, lactose, or aspartame, or a flavoring agent such as peppermint, oil of wintergreen, or cherry flavoring may be added.

When the unit dosage form is a capsule, it may contain, in addition to materials of the above type, a liquid carrier, such as a vegetable oil or a polyethylene glycol.

Various other materials may be present as coatings or for otherwise modifying the physical form of the solid unit dosage form. For instance, tablets, pills, or capsules may be coated with gelatin, wax, shellac, or sugar, and the like. A syrup or elixir may contain the active antibiotic, sucrose or fructose as a sweetening agent, methyl and propylparabens as preservatives, a dye, and flavoring such as cherry or orange flavor.

Any material used in preparing any unit dosage form should be pharmaceutically acceptable and substantially non-toxic in the amounts employed.

In addition, the antibiotics may be incorporated into sustained-release preparations and devices. For example, the antibiotics may be incorporated into time release capsules, time release tablets, time release pills, and time release antibiotics or nanoparticles.

Pharmaceutical compositions for topical administration of the antibiotics to the epidermis (mucosal or cutaneous surfaces) can be formulated as ointments, creams, lotions, gels, or as a transdermal patch. Such transdermal patches can contain penetration enhancers such as linalool, carvacrol, thymol, citral, menthol, t-anethole, and the like. Ointments and creams can, for example, include an aqueous or oily base with the addition of suitable thickening agents, gelling agents, colorants, and the like. Lotions and creams can include an aqueous or oily base and typically also contain one or more emulsifying agents, stabilizing agents, dispersing agents, suspending agents, thickening agents, coloring agents, and the like. Gels preferably include an aqueous carrier base and include a gelling agent such as cross-linked polyacrylic acid antibiotic, a derivatized polysaccharide (e.g., carboxymethyl cellulose), and the like.

Pharmaceutical compositions suitable for topical administration in the mouth (e.g., buccal or sublingual administration) include lozenges comprising the composition in a flavored base, such as sucrose, acacia, or tragacanth; pastilles comprising the composition in an inert base such as gelatin and glycerin or sucrose and acacia; and mouthwashes comprising the active ingredient in a suitable liquid carrier. The pharmaceutical compositions for topical administration in the mouth can include penetration enhancing agents, if desired.

Useful solid carriers include finely divided solids such as talc, clay, microcrystalline cellulose, silica, alumina, and the like. Other solid carriers include nontoxic polymeric nanoparticles or microparticles. Useful liquid carriers include water, alcohols, or glycols, or water/alcohol/glycol blends, in which the antibiotics can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the antibiotics to the skin are known in the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

The concentration of the therapeutic antibiotics of the invention in such formulations can vary widely depending on the nature of the formulation and intended route of administration. For example, the concentration of the antibiotics in a liquid composition, such as a lotion, can preferably be from about 0.1-25% by weight, or, more preferably, from about 0.5-10% by weight. The concentration in a semi-solid or solid composition such as a gel or a powder can preferably be about 0.1-5% by weight, or, more preferably, about 0.5-2.5% by weight.

Pharmaceutical compositions for spinal administration or injection into amniotic fluid can be provided in unit dose form in ampoules, pre-filled syringes, small volume infusion, or in multi-dose containers, and can include an added preservative. The compositions for parenteral administration can be suspensions, solutions, or emulsions, and can contain excipients such as suspending agents, stabilizing agents, and dispersing agents.

A pharmaceutical composition suitable for rectal administration comprises antibiotics of the present invention in combination with a solid or semisolid (e.g., cream or paste) carrier or vehicle. For example, such rectal compositions can be provided as unit dose suppositories. Suitable carriers or vehicles include cocoa butter and other materials commonly used in the art.

According to one embodiment, pharmaceutical compositions of the present invention suitable for vaginal administration are provided as pessaries, tampons, creams, gels, pastes, foams, or sprays containing antibiotics of the invention in combination with carriers as are known in the art. Alternatively, compositions suitable for vaginal administration can be delivered in a liquid or solid dosage form.

Pharmaceutical compositions suitable for intra-nasal administration are also encompassed by the present invention. Such intra-nasal compositions comprise antibiotics of the invention in a vehicle and suitable administration device to deliver a liquid spray, dispersible powder, or drops. Drops may be formulated with an aqueous or non-aqueous base also comprising one or more dispersing agents, solubilizing agents, or suspending agents. Liquid sprays are conveniently delivered from a pressurized pack, an insufflator, a nebulizer, or other convenient means of delivering an aerosol comprising the antibiotics. Pressurized packs comprise a suitable propellant such as dichlorodifluoromethane, trichlorofluoromethane, dichlorotetrafluoroethane, carbon dioxide, or other suitable gas as is well known in the art. Aerosol dosages can be controlled by providing a valve to deliver a metered amount of the antibiotic.

The antibiotics may be combined with an inert powdered carrier and inhaled by the subject or insufflated.

Pharmaceutical compositions for administration by inhalation or insufflation can be provided in the form of a dry powder composition, for example, a powder mix of the antibiotics and a suitable powder base such as lactose or starch. Such powder composition can be provided in unit dosage form, for example, in capsules, cartridges, gelatin packs, or blister packs, from which the powder can be administered with the aid of an inhalator or insufflator.

The exact amount (effective dose) of the antibiotic will vary from subject to subject, depending on, for example, the species, age, weight, and general or clinical condition of the subject, the severity or mechanism of any infection being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. Methods for the extrapolation of effective dosages in mice and other animals to humans are known to the art; for example, see U.S. Pat. No. 4,938,949, which is hereby incorporated by reference. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

In general, however, a suitable dose will be in the range of from about 0.001 to about 100 mg/kg of body weight per day, preferably from about 0.01 to about 100 mg/kg of body weight per day, more preferably, from about 0.1 to about 50 mg/kg of body weight per day, or even more preferred, in a range of from about 1 to about 10 mg/kg of body weight per day. For example, a suitable dose may be about 1 mg/kg, 10 mg/kg, or 50 mg/kg of body weight per day.

The antibiotics can be conveniently administered in unit dosage form, containing for example, about 0.05 to about 10000 mg, about 0.5 to about 10000 mg, about 5 to about 1000 mg, or about 50 to about 500 mg of active ingredient per unit dosage form.

The antibiotics can be administered to achieve peak plasma concentrations of, for example, from about 0.25 to about 200 μM, about 0.5 to about 75 μM, about 1 to about 50 μM, about 2 to about 30 μM, or about 5 to about 25 μM. Exemplary desirable plasma concentrations include at least 0.25, 0.5, 1, 5, 10, 25, 50, 75, 100 or 200 μM. For example, plasma levels may be from about 1 to about 100 micromolar or from about 10 to about 25 micromolar. This may be achieved, for example, by the intravenous injection of a 0.05 to 5% solution of the antibiotics, optionally in saline, or orally administered as a bolus containing about 1 to about 100 mg of the antibiotics. Desirable blood levels may be maintained by continuous or intermittent infusion.

The antibiotics can be included in the compositions within a therapeutically useful and effective concentration range, as determined by routine methods that are well known in the medical and pharmaceutical arts. For example, a typical composition can include one or more of the antibiotics at a concentration in the range of at least about 1 mg/ml, preferably at least about 4 mg/ml, more preferably at least 5 mg/ml and most preferably at least 6 mg/ml.

The antibiotics may conveniently be presented in a single dose or as divided doses administered at appropriate intervals, for example, as one dose per day or as two, three, four or more sub-doses per day. The sub-dose itself may be further divided, e.g., into a number of discrete loosely spaced administrations; such as multiple inhalations from an insufflator.

Optionally, the pharmaceutical compositions of the present invention can include one or more other therapeutic agents, e.g., as a combination therapy. The additional therapeutic agent(s) will be included in the compositions within a therapeutically useful and effective concentration range, as determined by routine methods that are well known in the medical and pharmaceutical arts. The concentration of any particular additional therapeutic agent may be in the same range as is typical for use of that agent as a monotherapy, or the concentration may be lower than a typical monotherapy concentration if there is a synergy when combined with antibiotics of the present invention.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

### 6. EXAMPLES

The following are examples that illustrate embodiments for practicing the disclosure described herein. These examples should not be construed as limiting.

All commercially available materials were used without further purification Amino acids, coupling reagents and resins were obtained from GL Biochem, CS Bio and Chem-Impex unless otherwise specified. All solvents were reagent grade or HPLC grade (RCI or DUKSAN). Anhydrous solvents were either prepared from AR grade solvents via standard methods (CH₂Cl₂, THF and CH₃CN), or purchased in anhydrous form (DMF, 1,2-dichloroethane).

### Example 1

### Synthesis of EpoxyC14-CDA3a

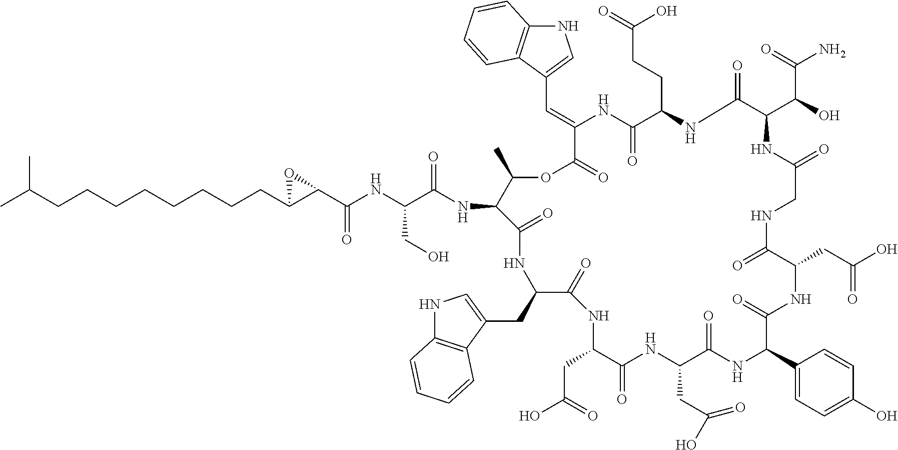 [SMILES: CC(C)CCCCCCCC[C@H]1O[C@@H]1C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The 2-formylphenyl (2S,3R)-3-(9-methyldecyl)oxirane-2-carboxylate was synthesized as follows: ethyl (E)-12-methyltridec-2-enoate (1.0 eq) was dissolved in anhydrous Et₂O was added DIBAL-H (2.5 eq). the reaction mixture was stirred at 0° C. for 2 h. then MeOH, diatomite and Na₂SO₄ was added. The reaction mixture was gradually warmed to rt, filtered through a short pad of Na₂SO₄, and the solid residue was washed by CH₂Cl₂. the removal of volatiles under reduced pressure and the crude residue was purified by flash chromatograph (hexane/EtOAc, 3:1) to yield pure (E)-12-methyltridec-2-en-1-ol as colourless oil. To a flame dried 50 mL round bottom flask filled with 4 Å powered molecular sieves (activated under high vacuum at 180° C. overnight), was added anhydrous CH₂Cl₂. the flask was cooled to −20° C. D-(−)-diethyl tartrate (0.075 eq) and Ti(OiPr)₄ (0.05 eq) were added sequentially via syringe. The mixture was stirred for 20 min before TBHP (2.0 eq) was introduced dropwise. The resulting mixture was stirred at −20° C. for 20 min. (E)-12-methyltridec-2-en-1-ol (1.0 eq), dried over molecular sieve for 2 h was dissolved in anhydrous CH₂Cl₂ and added dropwise via syringe over 40 min. the mixture was further stirred at −20° C. for 4 h monitored by TLC until completion. The reaction was quenched by adding sat. Na₂SO₃ at 0° C. and stirred for 3 h at rt. The cloudy suspension was filtered through Celite and washed with CH₂Cl₂. Hydrolysis of the tartrate complex in the filtrate was then effected by adding 30% aqueous NaOH solution saturated with NaCl and stirring vigorously for 30 min at 0° C. The aqueous layer was separated and washed with CH₂Cl₂. Combined organic layers were dried with anhydrous Na₂SO₄ and concentrated under reduced pressure. The yellow residue was purified by flash chromatography (hexane/EtOAc, 5:1) to afford the chiral ((2R,3R)-3-(9-methyldecyl)oxiran-2-yl)methanol.

A solution of ((2R,3R)-3-(9-methyldecyl)oxiran-2-yl)methanol (1.0 eq) in a system formed by CCl₄, CH₃CN and H₂O was treated with NaIO₄ (4.0 eq) and RuCl₃ (0.02 eq) at rt. After vigorous stirring for 1 h, a sat. NaHSO₃ was added, and after decantation, the aqueous phase was extracted with EtOAc. The combined organic extracts were dried via MgSO₄, filtered and concentrated under reduced pressure to afford the crude epoxy acid as a viscous black oil without purification.

A stirred solution of crude epoxy acid in anhydrous CH₂Cl₂, salicylic aldehyde (1.0 eq), DMAP (0.1 eq) and DIC (1.3 eq) were added sequentially. The reaction mixture was stirred at rt for 4 h. The reaction mixture was concentrated under reduced pressure and purified by flash chromatograph (hexane/EtOAc, 30:1) to afford the 2-formylphenyl (2S,3R)-3-(9-methyldecyl)oxirane-2-carboxylate as a colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained epoxyC14CDA3a.

### Example 2

### Synthesis of EpoxyC10-CDA3a

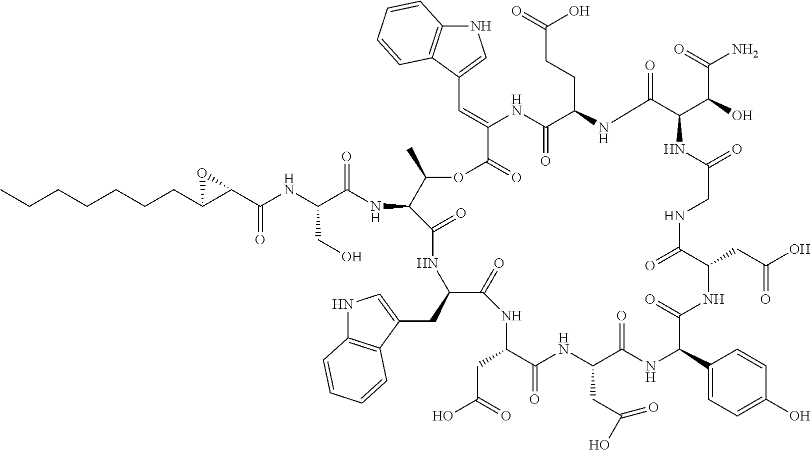 [SMILES: CCCCCCC[C@H]1O[C@@H]1C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The 2-formylphenyl (2S,3R)-3-heptyloxirane-2-carboxylate was synthesized as follows: ethyl (E)-dec-2-enoate (1.0 eq) was dissolved in anhydrous Et₂O was added DIBAL-H (2.5 eq). the reaction mixture was stirred at 0° C. for 2 h. then MeOH, diatomite and Na₂SO₄ was added. The reaction mixture was gradually warmed to rt, filtered through a short pad of Na₂SO₄, and the solid residue was washed by CH₂Cl₂. the removal of volatiles under reduced pressure and the crude residue was purified by flash chromatograph (hexane/EtOAc, 3:1) to yield pure (E)-dec-2-en-1-ol as colourless oil. To a flame dried 50 mL round bottom flask filled with 4 Å powered molecular sieves (activated under high vacuum at 180° C. overnight), was added anhydrous CH₂Cl₂. the flask was cooled to −20° C. D-(−)-diethyl tartrate (0.075 eq) and Ti(OiPr)₄ (0.05 eq) were added sequentially via syringe. The mixture was stirred for 20 min before TBHP (2.0 eq) was introduced dropwise. The resulting mixture was stirred at −20° C. for 20 min (E)-dec-2-en-1-ol (1.0 eq), dried over molecular sieve for 2 h was dissolved in anhydrous CH₂Cl₂ and added dropwise via syringe over 40 min the mixture was further stirred at −20° C. for 4 h monitored by TLC until completion. The reaction was quenched by adding sat. Na₂SO₃ at 0° C. and stirred for 3 h at rt. The cloudy suspension was filtered through Celite and washed with CH₂Cl₂. Hydrolysis of the tartrate complex in the filtrate was then effected by adding 30% aqueous NaOH solution saturated with NaCl and stirring vigorously for 30 min at 0° C. The aqueous layer was separated and washed with CH₂Cl₂. Combined organic layers were dried with anhydrous Na₂SO₄ and concentrated under reduced pressure. The yellow residue was purified by flash chromatography (hexane/EtOAc, 5:1) to afford the chiral ((2R,3R)-3-heptyloxiran-2-yl)methanol.

A solution of ((2R,3R)-3-heptyloxiran-2-yl)methanol (1.0 eq) in a system formed by CCl₄, CH₃CN and H₂O was treated with NaIO₄ (4.0 eq) and RuCl₃ (0.02 eq) at rt. After vigorous stirring for 1 h, a sat. NaHSO₃ was added, and after decantation, the aqueous phase was extracted with EtOAc. The combined organic extracts were dried via MgSO₄, filtered and concentrated under reduced pressure to afford the crude epoxy acid as a viscous black oil without purification.

A stirred solution of crude epoxy acid in anhydrous CH₂Cl₂, salicylic aldehyde (1.0 eq), DMAP (0.1 eq) and DIC (1.3 eq) were added sequentially. The reaction mixture was stirred at rt for 4 h. The reaction mixture was concentrated under reduced pressure and purified by flash chromatograph (hexane/EtOAc, 30:1) to afford the 2-formylphenyl (2S,3R)-3-heptyloxirane-2-carboxylate as a colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (20-60% CH₃CN/H₂O over 30 min) to obtained epoxyC10CDA3a.

### Example 3

### Synthesis of Δ^{2,3}-C10-CDA3a

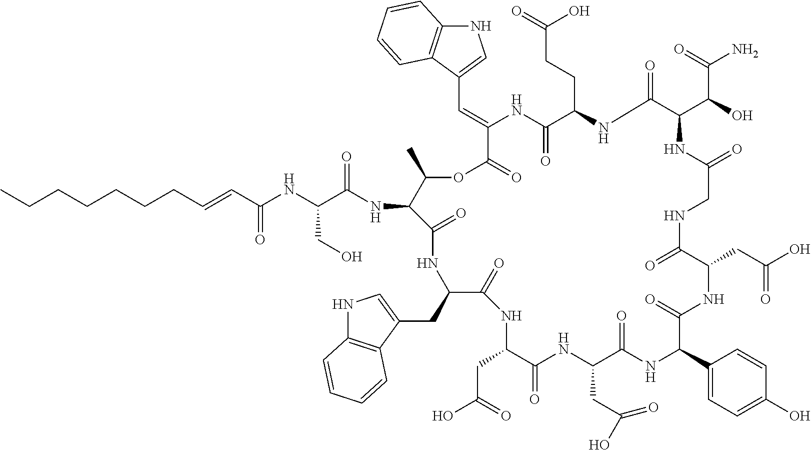 [SMILES: CCCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-dec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-dec-2-enoate as colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (20-60% CH₃CN/H₂O over 30 min) to obtained Δ^{2,3}-C10-CDA3a.

### Example 4

### Synthesis of Δ^{2,3}-C12-CDA3a

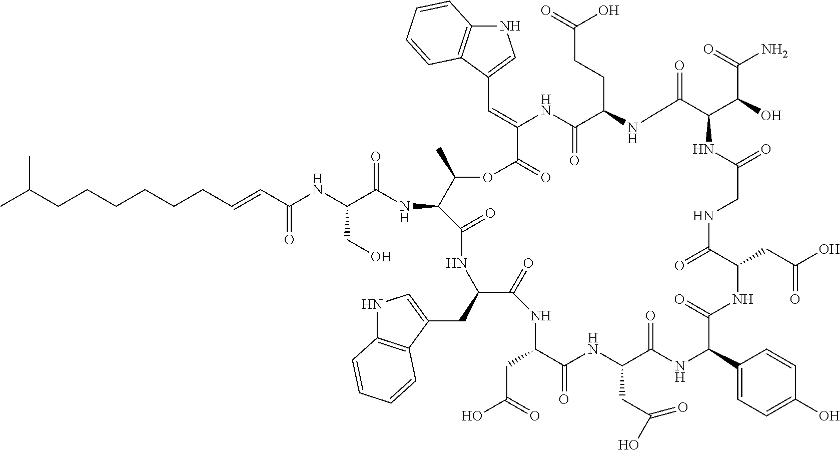 [SMILES: CC(C)CCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-10-methylundec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-10-methylundec-2-enoate as colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-70% CH₃CN/H₂O over 30 min) to obtained Δ^{2,3}-C12-CDA3a.

### Example 5

### Synthesis of Δ^{2,3}-C13-CDA3a

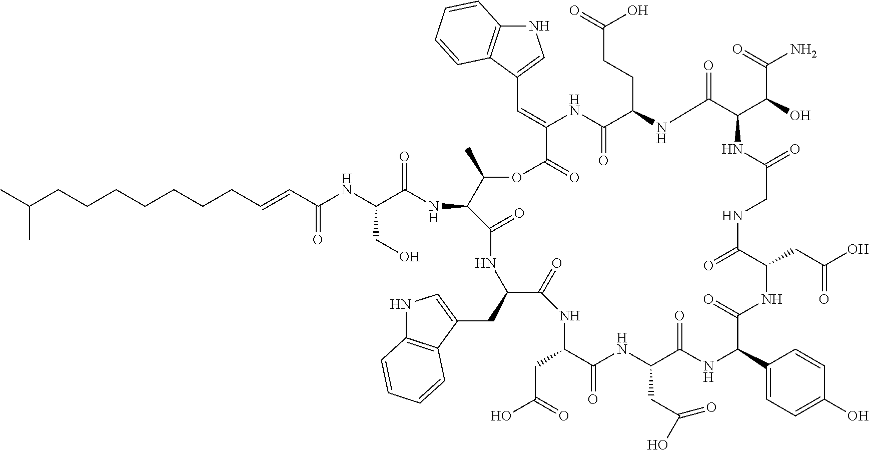 [SMILES: CC(C)CCCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-11-methyldodec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-11-methyldodec-2-enoateas colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained Δ^{2,3}-C13-CDA3a.

### Example 6

### Synthesis of Δ^{2,3}-C14-CDA3a

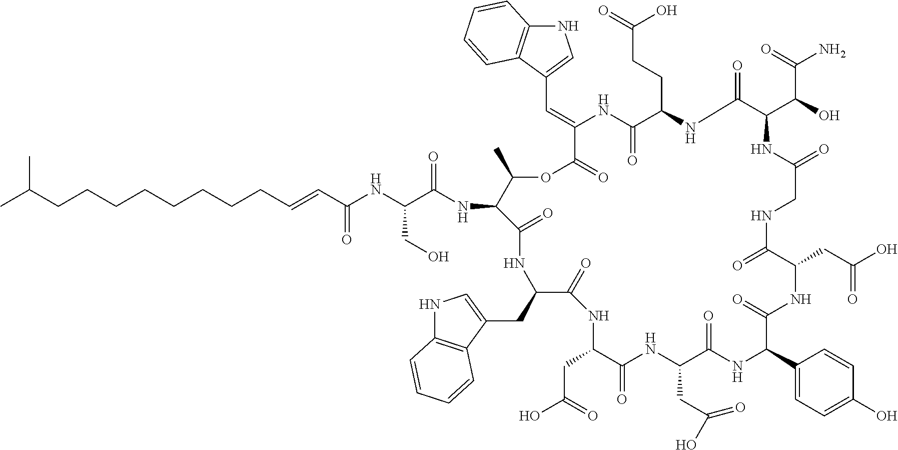 [SMILES: CC(C)CCCCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-12-methyltridec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-12-methyltridec-2-enoate as colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained Δ^{2,3}-C14-CDA3a.

### Example 7

### Synthesis of Δ^{2,3}-C15-CDA3a

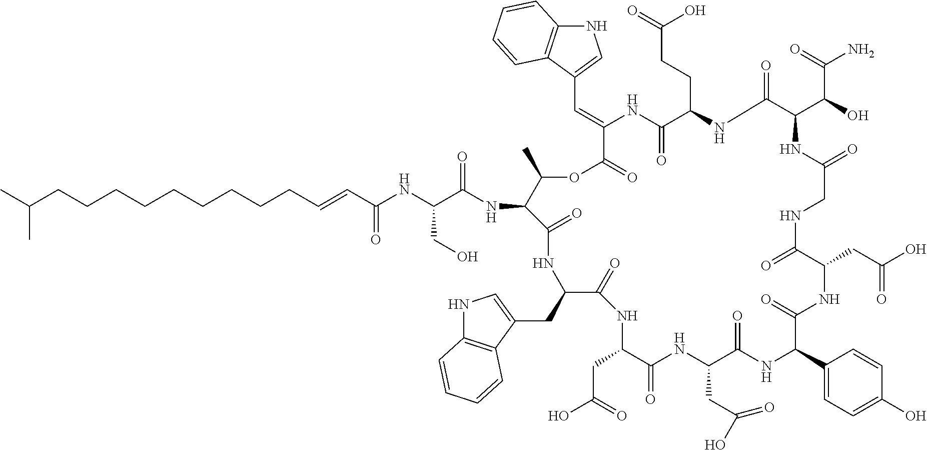 [SMILES: CC(C)CCCCCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-13-methyltetradec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-13-methyltetradec-2-enoate as colourless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-80% CH₃CN/H₂O over 30 min) to obtained Δ^{2,3}-C15-CDA3a.

### Example 8

### Synthesis of Δ^{2,3}-C16-CDA3a

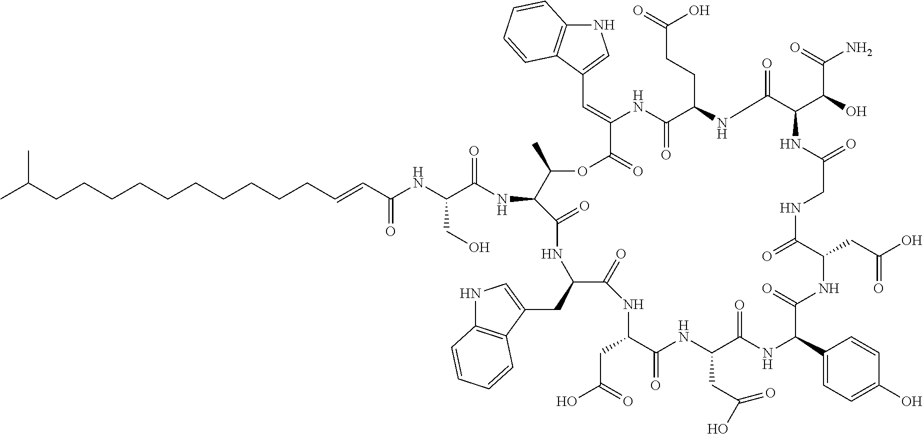 [SMILES: CC(C)CCCCCCCCCC/C=C/C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

A solution of ethyl (E)-14-methylpentadec-2-enoate (1.0 eq) in NaOH and tert-butyl alcohol (1:1, v/v) was stirred at 60° C. for 6 h. after being cooled to re, the reaction was acidified with 1N HCl and extracted with CH₂Cl₂. the combined organic extracts were washed with brine, dried over MgSO₄, and concentrated to dryness to yield the α,β-unsaturated fatty acids, which were used without further purification.

A mixture of the above unsaturated fatty acid and salicylic aldehyde (1.0 eq), DMAP (0.1 eq) and DIC (1.3 eq) in CH₂Cl₂ was stirred at rt for 8 h. the solvent was removed under reduce pressure and the crude residue was purified by flash chromatograph (hexane/EtIAc, 10:1) to yield pure 2-formylphenyl (E)-14-methylpentadec-2-enoate as colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (20-60% CH₃CN/H₂O over 30 min) to obtained Δ^{2,3}-C16-CDA3a.

### Example 9

### Synthesis of Epoxy-C14-CDA3b

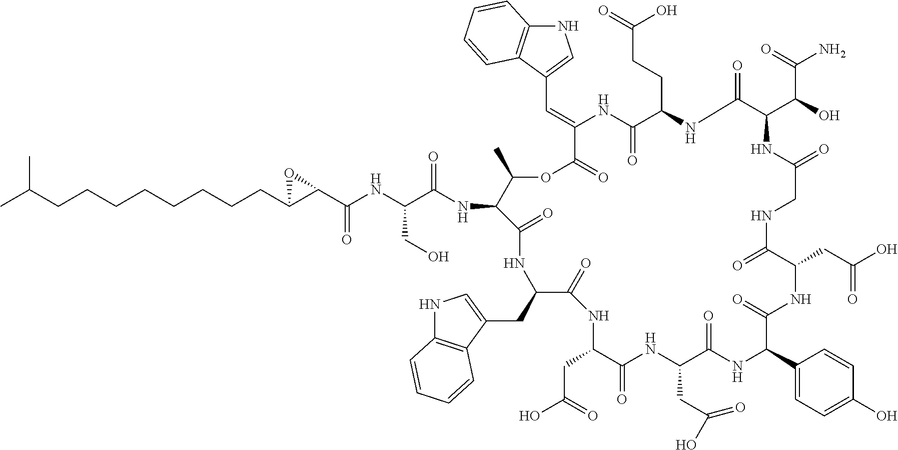 [SMILES: CC(C)CCCCCCCC[C@H]1O[C@@H]1C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-Trp-Glu-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The 2-formylphenyl (2S,3R)-3-(9-methyldecyl)oxirane-2-carboxylate was synthesized as follows: ethyl (E)-12-methyltridec-2-enoate (1.0 eq) was dissolved in anhydrous Et₂O was added DIBAL-H (2.5 eq). the reaction mixture was stirred at 0° C. for 2 h. then MeOH, diatomite and Na₂SO₄ was added. The reaction mixture was gradually warmed to rt, filtered through a short pad of Na₂SO₄, and the solid residue was washed by CH₂Cl₂. the removal of volatiles under reduced pressure and the crude residue was purified by flash chromatograph (hexane/EtOAc, 3:1) to yield pure (E)-12-methyltridec-2-en-1-ol as colorless oil. To a flame dried round bottom flask filled with 4 Å powered molecular sieves (activated under high vacuum at 180° C. overnight), was added anhydrous CH₂Cl₂. the flask was cooled to −20° C. D-(−)-diethyl tartrate (0.075 eq) and Ti(OiPr)₄ (0.05 eq) were added sequentially via syringe. The mixture was stirred for 20 min before TBHP (2.0 eq) was introduced dropwise. The resulting mixture was stirred at −20° C. for 20 min. (E)-12-methyltridec-2-en-1-ol (1.0 eq), dried over molecular sieve for 2 h was dissolved in anhydrous CH₂Cl₂ and added dropwise via syringe over 40 min. the mixture was further stirred at −20° C. for 4 h monitored by TLC until completion. The reaction was quenched by adding sat. Na₂SO₃ at 0° C. and stirred for 3 h at rt. The cloudy suspension was filtered through Celite and washed with CH₂Cl₂. Hydrolysis of the tartrate complex in the filtrate was then effected by adding 30% aqueous NaOH solution saturated with NaCl and stirring vigorously for 30 min at 0° C. The aqueous layer was separated and washed with CH₂Cl₂. Combined organic layers were dried with anhydrous Na₂SO₄ and concentrated under reduced pressure. The yellow residue was purified by flash chromatography (hexane/EtOAc, 5:1) to afford the chiral ((2R,3R)-3-(9-methyldecyl)oxiran-2-yl)methanol.

A solution of ((2R,3R)-3-(9-methyldecyl)oxiran-2-yl)methanol (1.0 eq) in a system formed by CCl₄, CH₃CN and H₂O was treated with NaIO₄ (4.0 eq) and RuCl₃ (0.02 eq) at rt. After vigorous stirring for 1 h, a sat. NaHSO₃ was added, and after decantation, the aqueous phase was extracted with EtOAc. The combined organic extracts were dried via MgSO₄, filtered and concentrated under reduced pressure to afford the crude epoxy acid as a viscous black oil without purification.

A stirred solution of crude epoxy acid in anhydrous CH₂Cl₂, salicylic aldehyde (1.0 eq), DMAP (0.1 eq) and DIC (1.3 eq) were added sequentially. The reaction mixture was stirred at rt for 4 h. The reaction mixture was concentrated under reduced pressure and purified by flash chromatograph (hexane/EtOAc, 30:1) to afford the 2-formylphenyl (2S,3R)-3-(9-methyldecyl)oxirane-2-carboxylate as a colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained epoxyC14CDA3b.

### Example 10

### Synthesis of Epoxy-C14-CDA4a

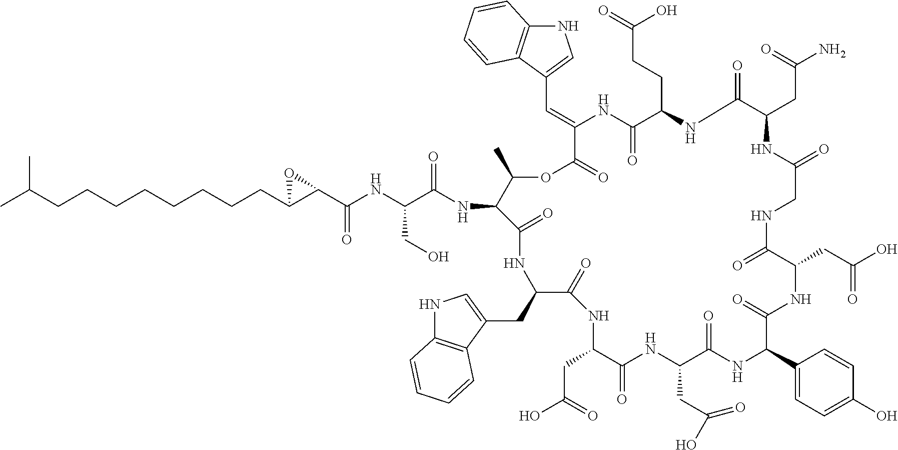 [SMILES: CC(C)CCCCCCCC[C@H]1O[C@@H]1C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H](CC(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-D-Asp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The 2-formylphenyl (2S,3R)-3-(9-methyldecyl)oxirane-2-carboxylate was synthesized as follows: ethyl (E)-12-methyltridec-2-enoate (1.0 eq) was dissolved in anhydrous Et₂O was added DIBAL-H (2.5 eq). the reaction mixture was stirred at 0° C. for 2 h. then MeOH, diatomite and Na₂SO₄ was added. The reaction mixture was gradually warmed to rt, filtered through a short pad of Na₂SO₄, and the solid residue was washed by CH₂Cl₂. the removal of volatiles under reduced pressure and the crude residue was purified by flash chromatograph (hexane/EtOAc, 3:1) to yield pure (E)-12-methyltridec-2-en-1-ol as colorless oil. To a flame dried 50 mL round bottom flask filled with 4 Å powered molecular sieves (activated under high vacuum at 180° C. overnight), was added anhydrous CH₂Cl₂. the flask was cooled to −20° C. D-(−)-diethyl tartrate (0.075 eq) and Ti(OiPr)₄ (0.05 eq) were added sequentially via syringe. The mixture was stirred for 20 min before TBHP (2.0 eq) was introduced dropwise. The resulting mixture was stirred at −20° C. for 20 min. (E)-12-methyltridec-2-en-1-ol (1.0 eq), dried over molecular sieve for 2 h was dissolved in anhydrous CH₂Cl₂ and added dropwise via syringe over 40 min. the mixture was further stirred at −20° C. for 4 h monitored by TLC until completion. The reaction was quenched by adding sat. Na₂SO₃ at 0° C. and stirred for 3 h at rt. The cloudy suspension was filtered through Celite and washed with CH₂Cl₂. Hydrolysis of the tartrate complex in the filtrate was then effected by adding 30% aqueous NaOH solution saturated with NaCl and stirring vigorously for 30 min at 0° C. The aqueous layer was separated and washed with CH₂Cl₂. Combined organic layers were dried with anhydrous Na₂SO₄ and concentrated under reduced pressure. The yellow residue was purified by flash chromatography (hexane/EtOAc, 5:1) to afford the chiral ((2R,3R)-3-(9-methyldecyl)oxiran-2-yl)methanol.

A solution of ((2R,3R)-3-(9-methyldecyl)oxiran-2-yl)methanol (1.0 eq) in a system formed by CCl₄ (3 mL), CH₃CN (3 mL) and H₂O (3 mL) was treated with NaIO₄ (4.0 eq) and RuCl₃ (0.02 eq) at rt. After vigorous stirring for 1 h, a sat. NaHSO₃ was added, and after decantation, the aqueous phase was extracted with EtOAc. The combined organic extracts were dried via MgSO₄, filtered and concentrated under reduced pressure to afford the crude epoxy acid as a viscous black oil without purification.

A stirred solution of crude epoxy acid in anhydrous CH₂Cl₂, salicylic aldehyde (1.0 eq), DMAP (0.1 eq) and DIC (1.3 eq) were added sequentially. The reaction mixture was stirred at rt for 4 h. The reaction mixture was concentrated under reduced pressure and purified by flash chromatograph (hexane/EtOAc, 30:1) to afford the 2-formylphenyl (2S,3R)-3-(9-methyldecyl)oxirane-2-carboxylate as a colourless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, 1 mL TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained epoxyC14CDA4a.

### Example 11

### Synthesis of Epoxy-C14-Epi-D-HOAsn-CDA3a

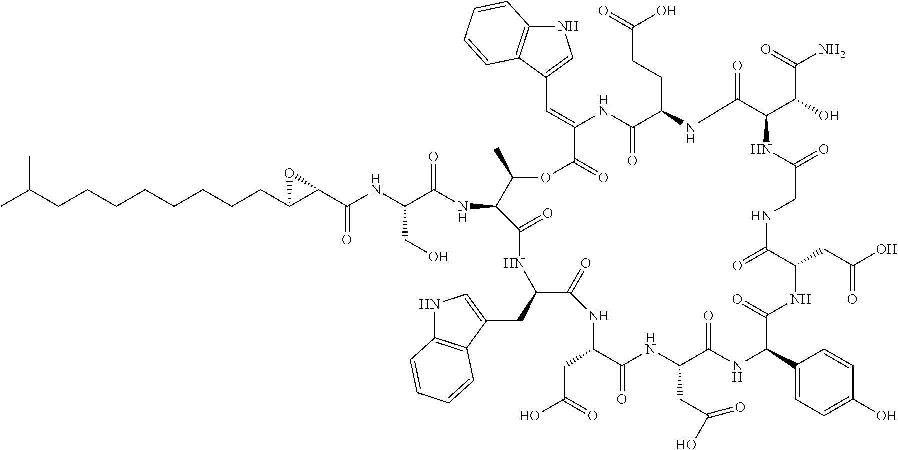 [SMILES: CC(C)CCCCCCCC[C@H]1O[C@@H]1C(=O)N[C@@H](CO)C(=O)N[C@@H]1C(=O)N[C@H](Cc2c[nH]c3ccccc23)C(=O)N[C@@H](CC(=O)O)C(=O)N[C@@H](CC(=O)O)C(=O)N[C@H](c2ccc(O)cc2)C(=O)N[C@@H](CC(=O)O)C(=O)NCC(=O)N[C@H]([C@@H](O)C(N)=O)C(=O)N[C@H](CCC(=O)O)C(=O)N/C(=C\c2c[nH]c3ccccc23)C(=O)O[C@@H]1C]

Linear peptide resin-Gly-Asp-D-Hpg-Asp-Asp-D-Trp-Thr[O-ΔTrp-Glu-epi-D-HOAsp-NH₂]-Ser(OtBu)NHBoc was synthesized by 9H-fluoren-9-yl-methoxycarbonyl (Fmoc) solid phase peptide synthesis protocol. The peptide was cleaved from the 2-chlorotrityl resin under mild conditions (TFE/AcOH/DCM). After drying, the peptide was cyclized using HATU/HOAt/DIEA in CH₂Cl₂ (dichloromethane, DCM) at a concentration of 0.1 mM for 24 h at room temperature. CH₂Cl₂ was evaporated under low pressure and the residue was treated with a mixture of 5 mL TFA/phenol/H₂O (v/v/v=95:2.5:2.5) for 1.5 hours. The mixture of TFA/phenol/H₂O was blown off by a condensed air stream and residue was washed by ethyl ether. The residue was purified by preparative HPLC (5-50% CH₃CN/H₂O over 30 min) to afford the side chain unprotected cyclic peptide.

The 2-formylphenyl (2S,3R)-3-(9-methyldecyl)oxirane-2-carboxylate was synthesized as follows: ethyl (E)-12-methyltridec-2-enoate (1.0 eq) was dissolved in anhydrous Et₂O was added DIBAL-H (2.5 eq). the reaction mixture was stirred at 0° C. for 2 h. then MeOH, diatomite and Na₂SO₄ was added. The reaction mixture was gradually warmed to rt, filtered through a short pad of Na₂SO₄, and the solid residue was washed by CH₂Cl₂. the removal of volatiles under reduced pressure and the crude residue was purified by flash chromatograph (hexane/EtOAc, 3:1) to yield pure (E)-12-methyltridec-2-en-1-ol as colorless oil. To a flame dried 50 mL round bottom flask filled with 4 Å powered molecular sieves (activated under high vacuum at 180° C. overnight), was added anhydrous CH₂Cl₂. The flask was cooled to −20° C. D-(−)-diethyl tartrate (0.075 eq) and Ti(OiPr)₄ (0.05 eq) were added sequentially via syringe. The mixture was stirred for 20 min before TBHP (2.0 eq) was introduced dropwise. The resulting mixture was stirred at −20° C. for 20 min (E)-12-methyltridec-2-en-1-ol (1.0 eq), dried over molecular sieve for 2 h was dissolved in anhydrous CH₂Cl₂ and added dropwise via syringe over 40 min the mixture was further stirred at −20° C. for 4 h monitored by TLC until completion. The reaction was quenched by adding sat. Na₂SO₃ at 0° C. and stirred for 3 h at rt. The cloudy suspension was filtered through Celite and washed with CH₂Cl₂. Hydrolysis of the tartrate complex in the filtrate was then effected by adding 30% aqueous NaOH solution saturated with NaCl and stirring vigorously for 30 min at 0° C. The aqueous layer was separated and washed with CH₂Cl₂. Combined organic layers were dried with anhydrous Na₂SO₄ and concentrated under reduced pressure. The yellow residue was purified by flash chromatography (hexane/EtOAc, 5:1) to afford the chiral ((2R,3R)-3-(9-methyldecyl)oxiran-2-yl)methanol.

A solution of ((2R,3R)-3-(9-methyldecyl)oxiran-2-yl)methanol (1.0 eq) in a system formed by CCl₄ (3 mL), CH₃CN (3 mL) and H₂O (3 mL) was treated with NaIO₄ (4.0 eq) and RuCl₃ (0.02 eq) at rt. After vigorous stirring for 1 h, a sat. NaHSO₃ was added, and after decantation, the aqueous phase was extracted with EtOAc. The combined organic extracts were dried via MgSO₄, filtered and concentrated under reduced pressure to afford the crude epoxy acid as a viscous black oil without purification.

A stirred solution of crude epoxy acid in anhydrous CH₂Cl₂, salicylic aldehyde (1.0 eq), DMAP (0.1 eq) and DIC (1.3 eq) were added sequentially. The reaction mixture was stirred at rt for 4 h. The reaction mixture was concentrated under reduced pressure and purified by flash chromatograph (hexane/EtOAc, 30:1) to afford the 2-formylphenyl (2S,3R)-3-(9-methyldecyl)oxirane-2-carboxylate as a colorless oil.

The resulting salicylaldehyde ester and the cyclic peptide were dissolved in a mixture of pyridine/AcOH (mole/mole=1:1) and stirred at room temperature for 10 hours. After the solvent was removed by lyophilization, TFA/H₂O/AcOH (v/v/v=1:4:4) was added and stirred for 1 hour. TFA/H₂O/AcOH was blown away by a condensed air stream. The residue was purified by preparative HPLC (30-75% CH₃CN/H₂O over 30 min) to obtained epoxy-C14-epi-D-HOAsn-CDA3a.

### Example 12

### Antibacterial Studies

S. aureus and E. faecalis strains were assayed for their susceptibilities to both daptomycin and analogues respectively by the broth microdilution method as recommended by the clinical and laboratory standards institute (CLSI) in the presence of 8 mM calcium chloride. MIC (minimal inhibitory concentration) was recorded at which no bacterial growth was observed. For calcium dependence assay, standard procedure of susceptibility test was performed as above except in the presence of gradient concentrations of calcium chloride (0, 0.125, 0.25, 0.5, 1, 2, 4, 8 mM).

### Example 13

### Cell Permeability

A 1:100 dilution of an overnight culture of S. aureus strain ATCC29213 in MHB was incubated at 37° C. with constant aeration until reaching OD600 values of 0.5-0.6. Bacteria were treated with compounds at concentrations 0.5 μg/ml and incubated at 37° C. and 250 rpm. Treatment conditions determined according to the time-kill assay when killing effect was not detectable. At intervals of 0.5 h, aliquots were removed and washed three times with sterile saline. 200 μl of bacteria suspension were incubated with 1 μM SYTOX Green for 10 minutes at room temperature. Fluorescence was measured by a microplate reader (CLARIOstar, BMG) at a wavelength of 523±5 nm upon excitation at 504±5 nm.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

What is claimed:

1. An antibacterial compound represented by the structure of formula (I): 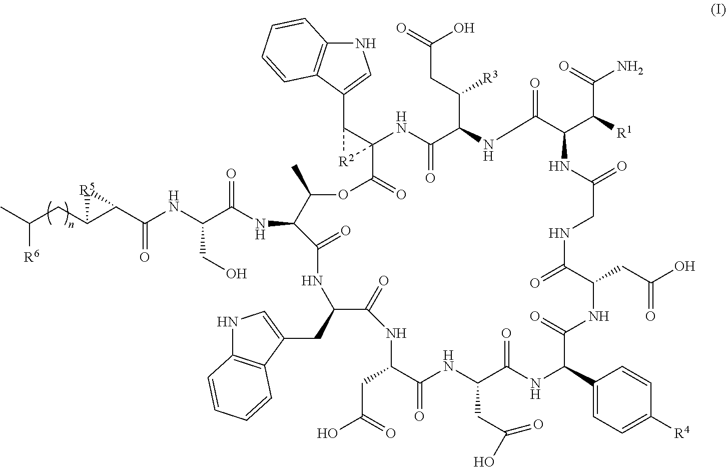
wherein:
R¹ is selected from the group consisting of H, OH and OPO₃H₂;
R² is selected from the group consisting of H, H and a π bond;
R³ is selected from the group consisting of H and CH₃;
R⁴ is selected from the group consisting of H, OH and halogen;
R⁵ is selected from the group consisting of O and a π bond
R⁶ is selected from the group consisting of H and CH₃.
n is an integer from 3-12;
and pharmaceutically acceptable salts thereof.

2. The compound according to claim 1, wherein R¹ is H.

3. The compound according to claim 1, wherein R¹ is OH.

4. The compound according to claim 1, wherein R² is H,H.

5. The compound according to claim 1, wherein R² is a π bond.

6. The compound according to claim 1, wherein R³ is H.

7. The compound according to claim 1, wherein R⁴ is OH.

8. The compound according to claim 1, wherein R⁵ is O.

9. The compound according to claim 1, wherein R⁵ is a π bond.

10. The compound according to claim 1, wherein n is an integer from 3 to 12.

11. The compound according to claim 1, wherein R⁶ is H.

12. The compound according to claim 1, wherein R⁶ is CH₃.

13. The compound according to claim 1, wherein
(l) R¹ is 3(S)—OH, R² is a π bond; R³ is H, R⁴ is OH, and R⁵ is a π bond, R⁶ is H, wherein n is 5; [Δ^{2,3}-C10-CDA3a]
(m) R¹ is 3(S)—OH, R² is a π bond; R³ is H, R⁴ is OH, and R⁵ is a π bond, R⁶ is CH₃, wherein n is 6; [Δ^{2,3}-C12-CDA3a]
(n) R¹ is 3(S)—OH, R² is a π bond; R³ is H, R⁴ is OH, and R⁵ is a π bond, R⁶ is CH₃, wherein m is 7; [Δ^{2,3}-C13-CDA3a]
(o) R¹ is 3(S)—OH, R² is a π bond; R³ is H, R⁴ is OH, and R⁵ is a π bond, R⁶ is CH₃, wherein m is 8; [Δ^{2,3}-C14-CDA3a]
(p) R¹ is 3(S)—OH, R² is a π bond; R³ is H, R⁴ is OH, and R⁵ is a π bond, R⁶ is CH₃, wherein m is 9; [Δ^{2,3}-C15-CDA3a]
(q) R¹ is 3(S)—OH, R² is a π bond; R³ is H, R⁴ is OH, and R⁵ is a π bond, R⁶ is CH₃, wherein m is 10; [Δ^{2,3}-C16-CDA3a]
(r) R¹ is 3(S)—OH, R² is a π bond; R³ is H, R⁴ is OH, and R⁵ is O, R⁶ is H, wherein n is 5; [epoxyC10-CDA3]
(s) R¹ is 3(S)—OH, R² is a π bond; R³ is H, R⁴ is OH, and R⁵ is O, R⁶ is CH₃, wherein m is 8; [epoxyC14-CDA3a]
(t) R¹ is 3(R)—OH, R² is a π bond; R³ is H, R⁴ is OH, and R⁵ is O, R⁶ is CH₃, wherein m is 8; [epoxy-C14-epi-D-HOAsn-CDA3a]
(u) R¹ is H, R² is a π bond; R³ is H, R⁴ is OH, and R⁵ is O, R⁶ is CH₃, wherein m is 8; [epoxy-C14-CDA4a]; or
(v) R¹ is 3(S)—OH, R² H, H; R³ is H, R⁴ is OH, and R⁵ is O, R⁶ is CH₃, wherein m is 8; [epoxy-C14-CDA3b];
and a pharmaceutically acceptable salts thereof.

14. The compound according to claim 1, wherein the compound is a CDA-3a antibiotic analogue.

15. An anti-bacterial pharmaceutical composition comprising a therapeutically effective amount of a compound of claim 1, and a pharmaceutically acceptable carrier or excipient.

16. A method of inhibiting growth of a bacteria comprising the step of contacting the bacteria with a compound according to claim 1.

17. A method of treating a bacterial infection in a subject, comprising the step of administering a compound according to claim 1 to the subject in need thereof.

18. The method according to claim 14 wherein the bacteria is a gram-positive bacteria.

19. The method according to claim 14 wherein the bacteria is a gram-negative bacteria.

20. The method according to claim 14 wherein the bacteria is a methicillin-resistant bacteria.

21. The method according to claim 14 wherein the bacteria is a vancomycin-resistant bacteria.
